(19) United States
(12) Patent Application Publication
(10) Pub. No.: US 2009/0287243 A1
(43) Pub. Date: Nov. 19, 2009

(54) EXTERNAL COUNTERPULSATION DEVICE AND METHOD

(76) Inventor: Shai Greennberg, Herzlia (IL)

Correspondence Address: BROOKS KUSHMAN P.C., 1000 TOWN CENTER, TWENTY-SECOND FLOOR, SOUTHFIELD, MI, 48075, US

(21) Appl. No.: 11/720,888

(22) PCT Filed: Dec. 5, 2005
(86) PCT No.: PCT/IL05/01305

§ 371 Date: Jun. 5, 2007

(30) Foreign Application Priority Data
Dec. 7, 2004 (IL) 165596

(51) Int. Cl. A61B 17/00; A61N 1/04; A61B 5/01

(52) U.S. Cl. 606/202; 607/115; 600/549

(57) ABSTRACT

An apparatus for improving heart perfusion of a subject is provided. The apparatus includes at least one device designed capable of periodically reducing blood flow in at least one appendage of the subject; and a control unit for controlling an operation of the at least one device.

### FIELD AND BACKGROUND OF THE INVENTION

The present invention relates to an apparatus and method for treating cardiovascular diseases and in particular ischemic heart disease and related disorders.

Cardiovascular disease is the leading cause of death in the U.S., according to CDC statistics. Conventional treatments include changes in lifestyle (e.g. dieting exercise), prescription drugs and surgery. While often effective at relieving symptoms such as chest pain or angina, these approaches are frequently ineffective at extending life, are associated with numerous side effects and complications, and are expensive. Heart surgery carries a significant risk of death, and often leads to other complications. In addition, a significant number of patients treated with drugs or surgery exhibit symptoms of the disease with time; following bypass surgery, it is estimated that only 75% of patients remain free of cardiac ischemia for five years, a figure that drops to 50% by ten years.

In recent years, External Counterpulsation (ECP) or Enhanced External Counterpulsation (EECP) has gained acceptance as an effective treatment modality for cardiovascular diseases.

Though ECP is a non-invasive treatment approach, it finds it origins in an invasive device, the intra-aortic balloon pump. ECP has a similar mechanism to the intra-aortic balloon pump, but works on the outside of the body.

An ECP unit includes a computer microprocessor, which triggers the sequential inflation with compressed air of cuffs that are wrapped around a patient's calves, thighs and buttocks. Compression is triggered to occur during diastole (the resting phase of the heart rhythm). As the computer inflates the cuffs, blood is propelled from the lower body back into the heart. At the end of diastole, the ECP computer signals the sudden and simultaneous deflation of the cuffs, greatly reducing vascular resistance and assisting the heart with its next beat. This action also facilitates venous return of blood into the heart, increasing cardiac output. Typically, cuff pressure is adjusted according to a ratio between diastolic and systolic pressures as measured by finger plasmography during treatment.

ECP improves blood flow to the coronary arteries and helps treat angina, the indication for which it has received FDA approval. Research suggests that ECP helps angina by causing the release of a hormone known as vascular endothelial growth factor (VEGF), by increasing NO secretion or by restoring endothelial function to the coronary arteries. Since ECP causes the heart to receive an unexpected surge of blood during diastole (when the heart empties and coronary flow occurs), the levels of VEGF increase dramatically which in turn promotes the development of collateral coronary vessels. Although prior art ECP devices are effective in treatment and management of heart disease, such devices are bulky and require that the treated subject be immobilized on a bed for the duration of the treatment.

There is thus a widely recognized need for, and it would be highly advantageous to have, an ECP apparatus devoid of the above limitations.

### SUMMARY OF THE INVENTION

According to one aspect of the present invention there is provided an apparatus for improving heart perfusion of a subject comprising: (a) at least one device designed capable of periodically reducing blood flow in at least one appendage of the subject; and (b) a control unit being capable of calculating flow in peripheral arteries and being for controlling an operation of the at least one device according to the flow in the peripheral arteries.

According to features in the described preferred embodiments the at least one device is an inflatable cuff.

According to still further features in the described preferred embodiments the at least one device is designed for use on an arm of the subject.

According to still further features in the described preferred embodiments the at least one device includes an electrode for electrically stimulating contraction of a muscle of the at least one appendage.

According to still further features in the described preferred embodiments the at least one device includes a sensor for measuring blood flow and whereas the control unit is capable of calculating flow changes in peripheral arteries according to data received from the sensor.

According to another aspect of the present invention there is provided an apparatus for improving heart perfusion of a subject comprising: (a) at least one device designed capable of periodically reducing blood flow in at least one appendage of the subject; (b) a temperature sensor being for measuring a temperature of the at least one appendage; and (b) a control unit for controlling an operation of the at least one device according to temperature data received from the temperature sensor.

According to still further features in the described preferred embodiments the temperature sensor is integrated with the at least one device designed capable of periodically reducing blood flow to the at least one appendage of the subject.

According to still further features in the described preferred embodiments the at least one device is an inflatable cuff.

According to still further features in the described preferred embodiments the at least one device is designed for use on an arm of the subject.

According to still further features in the described preferred embodiments the at least one device includes an electrode for electrically stimulating contraction of a muscle of the at least one appendage.

According to yet another aspect of the present invention there is provided a method of improving heart perfusion comprising: (a) periodically reducing blood flow in at least one appendage of a subject; and (b) subjecting the subject to physical exercise, thereby improving heart perfusion of the subject.

According to still further features in the described preferred embodiments the at least one appendage is an arm.

According to still further features in the described preferred embodiments the physical exercise is walking or running.

According to still further features in the described preferred embodiments step (a) is effected by applying to the at least one appendage a pressure higher than a diastolic pressure of the subject for a predetermined period of time.

According to still further features in the described preferred embodiments steps (a) and (b) are effected simultaneously.

According to still another aspect of the present invention there is provided system for improving heart perfusion of a subject comprising: (a) at least one device designed capable of periodically reducing blood flow in at least one appendage of the subject; (b) an exercise unit for subjecting the subject to physical exercise; (c) a control unit for controlling an operation of the at least one device and the exercise unit.

According to still further features in the described preferred embodiments the at least one device is an inflatable cuff.

According to still further features in the described preferred embodiments the at least one device is designed for use on an arm of the subject.

According to still further features in the described preferred embodiments the at least one device includes an electrode for electrically stimulating contraction of a muscle of the at least one appendage.

According to still further features in the described preferred embodiments the exercise unit is selected from the group consisting of a stationary bicycle, a treadmill and an arm ergometer.

The present invention successfully addresses the shortcomings of the presently known configurations by providing a counterpulsation device and method which can be used while exercising and without requiring the need for complicated and expensive equipment.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a prior art Figure illustrating changes in Arterial pressure curves between the aortic arch and peripheral arteries. Bern and Levi Am J. Physiology 218; 1970.

FIG. 2 illustrates changes in arterial pulse curve between the aortic arch and the radial artery during different exercise intensity. As exercise intensity is increase, heart rate increases, the systolic pressure curve is sharpen and the diastolic portion is reduced.

FIG. 3a illustrates a counterpulsation apparatus constructed according to the teachings of the present invention

FIG. 3b illustrates a an exercise system which incorporates a counterpulsation apparatus.

FIG. 4 illustrates cuff designed for pressure gradient along, the cuff from the distal part to the proximal to insure retrograde flow during diastole.

FIGS. 5a-b illustrate skin temperature response to changes in external cuff pressure: As shown there is a direct correlation between applied cuff pressure (FIG. 5b) and temperature changes (FIG. 5a). At pressure just above diastolic pressure (blue line, FIG. 5b) temperature started to rise (FIG. 5a).

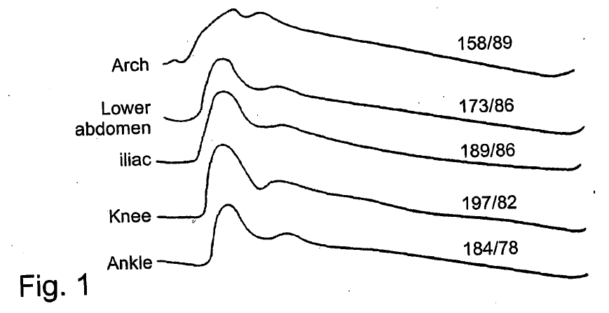

### DESCRIPTION OF THE PREFERRED EMBODIMENTS

The present invention is of an external counter pulsation apparatus and system which can be used to treat various cardiovascular diseases.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

External Counterpulsation (ECP) devices are well known in the art. Although various configurations of such devices are known, all utilize cuffs for applying pressure to the lower extremities and/or buttocks area, since it is believed that ECP diastolic augmentation requires pushing of blood towards the heart and restriction of blood flow to major blood pooling regions (e.g. legs, buttocks).

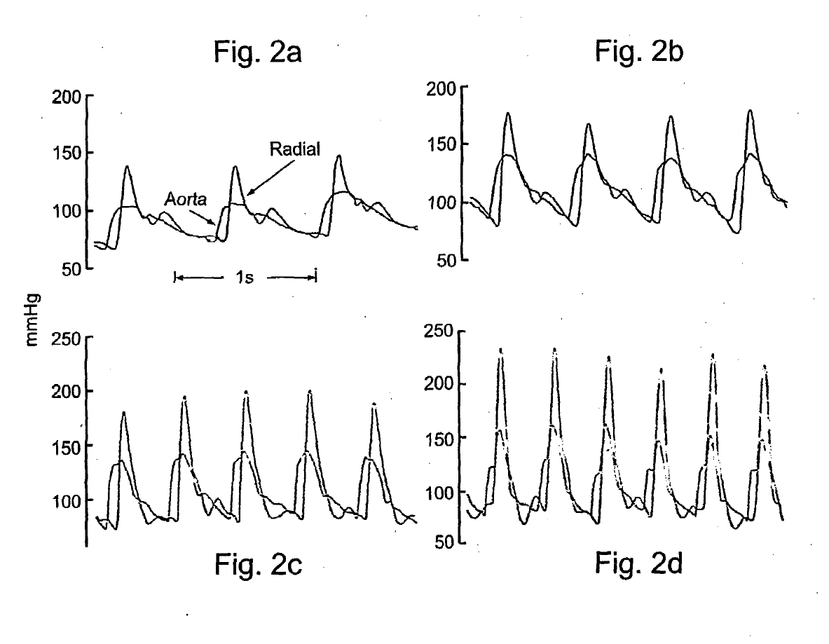

While reducing the present invention to practice, the present inventors hypothesized that ECP can also be effective using arm cuffs especially when ECP is applied in combination with exercising, since exercise significantly increases systole and decreases diastole (FIG. 2).

During exercise, metabolic demands of working muscles are increased thus requiring the body to adjust heart rate and blood pressure. The increase in the heart rate co-insides with an increase in exercise intensity; systolic blood pressure rises in order to increase the blood supply to the peripheral muscles, and a small decline in diastolic pressure occurs. Vasodilation response occurs in the arteries of the lower extremity working muscles. It has been found that in patients with cardiovascular risk factors, leg exercise induced a 5% increase in the diameter of the arteries and blood flow was increased 5 fold, from 250 ml/m during rest to about 1250 ml/m, due to the increase in systolic blood pressure. Blood flow to the arms also increases approximately 34 fold with no vasodilation response, from 100 ml/m to 350 ml/m (Ganzer J. Am. Coll. Cardiol. 2001; 38(5):1313-9IL).

Coronary blood flow is closely related to the cardiac workload. During exercise, the rise in systolic blood pressure causes an increase in the mean arterial pressure. As a result, there is an increase in coronary perfusion pressure which leads to a decrease in coronary artery resistance and an increase in blood flow to the coronaries.

Thus, according to one aspect of the present invention there is provided a method of improving heart perfusion in a subject. The subject can be a male or a female which suffers from, or is predisposed to, a cardiovascular disease such as a coronary artery disease, ischemic heart disease, angina pectoris, X syndrome, coronary vasospasm, chronic heart failure, pulmonary disease, diabetes, and peripheral arteries disease. The method is effected by periodically reducing blood flow in at least one appendage (e.g. arm and/or leg) and/or body portion (e.g. buttocks) while preferably subjecting the subject to physical exercise.

Reduction in blood flow can result in partial blood flow to the appendage or portion, or in no blood flow at all (complete restriction). Reduction in blood flow is effected periodically over the course of treatment in correlation with blood pressure of the subject and optionally other parameters such as skin temperature changes, changes in pulse pressure waves or Kortikov sounds. A blood flow reduction sequence including several blood flow reduction cycles can be effected according to known parameters.

Reducing blood flow to at least one appendage and/or body portion can be effected by any blood flow restricting device known in the art. Preferably, blood flow reduction is effected using pressure cuffs which are pneumatically or mechanically deployed. Examples of pressure cuffs include those available from ArtAssist ACI Medical USA.

Such cuffs can be manually operated by a skilled operator to produce the operational sequence desired. Preferably, such cuffs are attached to a control unit which controls operation of the cuffs including time and duration of deployment, and pressure applied by the cuff on the appendage. Control units attachable to pressure cuffs and capable of executing the parameters described above are well known in the art, examples include the WizAir® DVT system marketed by MCS Israel.

Reduction in blood flow can also be achieved by muscle contraction. During isometric muscle contraction the blood vessel near the contracting muscle is squeezed due to increase in transmural pressure and as a result blood flow in the arteries is reduced (Hamada et al. 2004). Muscle contraction can be effected voluntarily or via external electric stimulation. The latter can be effected via muscle stimulating electrodes (e.g. Zenith Electrotherapy MODEL ZS4—4 Channel MultiStim System Germany) which can be placed on an appendage (e.g. arm) and operated to stimulate contraction of biceps and/or triceps in correlation with blood pressure readings. Such isometric muscle stimulation will reduce blood flow in the brachial artery.

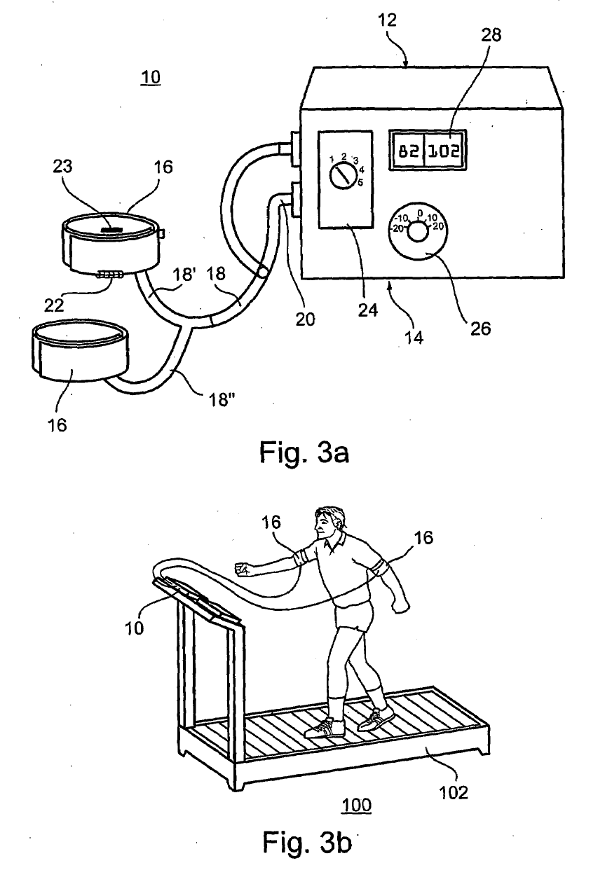

Preferably, blood flow reduction is effected by using a novel ECP apparatus which utilizes arm mounted cuffs and is further described hereinbelow with respect to FIGS. 3a-b.

The physical exercise performed by the subject is preferably selected/designed capable of increasing cardiovascular output and/or increasing blood flow to appendages of the subject, preferably appendages to which blood flow reduction is not applied. Exemplary physical exercises which can be used by the method of the present invention include walking, running, cycling, weight lifting stair climbing (using a stepper) cross training and arm ergometry.

Physical exercise is effected by using an exercise device such as a treadmill, a stationary cycle, arm ergometer a Stairmaster™ and the like. The subject can be subjected to an exercise routine prior to, following or preferably during a blood flow reduction sequence.

A typical exercise regimen can include a warm-up period of approximately 5 minutes at an exercise intensity of about 75% of the subject's target exercise level. Following 3-5 minutes of activity cuff pressure adjustment is performed. Cuffs are then deployed (e.g. inflated) for 5 sec to a pressure of 160 mmhg and then deflated to a pressure below diastolic pressure. Cuff pressure is first set to an inflation pressure about 10 to 20 mmHg below diastolic pressure, according to sensor signal measured during cuff inflation and deflation.

Exercise level is then increased to a target heart rate or a target training level. A second set of measurements/cuff pressure adjustment is then performed to set a pressure above the diastolic blood pressure of the subject.

Measurements are preformed at 5 minute intervals during the exercise session.

The upper/lower appendages are then exercised for five minutes without the use of the cuffs. This will magnify the blood flow to the brachial/femoral arteries, ensuring their function and the training effects on the extremities that do not participate in the exercise.

Although use of standard method parameters would benefit any individual suffering or predisposed to a cardiovascular disease, parameters such as: (i) time period of treatment; (ii) appendage or body portion subjected to blood flow reduction; (iii) extent of blood flow reduction; and (iv) type of physical exercise regimen are each preferably determined according to the type and severity of the cardiovascular disorder.

Typically, individuals suffering from coronary heart disease, should exercise 40-50 minutes, 5 times a week at an intensity which is 60-80% of maximal exercise intensity.

Thus, the present invention provides a novel approach for improving heart perfusion in a subject. Since increased heart perfusion has been correlated with treatment of various cardiovascular disorders, and as such the present method is highly suitable for use in treating such disorders. The following exemplifies use of the present method in treating ischemic and coronary heart and chronic heart disease.

Common treatment at cardiac rehabilitation and prevention center includes thirty six exercise sessions (3 per a week), wherein the exercise level is determined on the basis of an exercise test. Typically, exercise intensity during treatment is 60%-80% of maximal exercise levels.

Exercise sessions are supervised and are determined according to patients medical history. During exercise, patients are monitored via an ECG system and blood pressure measurements are performed during rest, exercise and recovery.

The present method also finds use in increasing cerebral perfusion during rest in subjects suffering from cerebral stroke or ischemia, or vasospasm of cerebral blood supply.

Studies have shown that thigh cuffs inflated to a pressure above systolic pressure significantly effect cerebral flow and increase both systolic and diastolic blood pressure by about 10% (Pinerai et al 2001). Furthermore, arm cuffs inflated above systolic pressure significantly change blood flow in cerebral arteries (Kashyap et al. 2005).

Based on these findings, employing the present method using thigh or thigh and arm cuffs at a pressure that is equal to the pressure that will occlude the femoral artery during diastole, will lead to an increase in cerebral flow during systole (increased lower body resistance will increase systolic blood pressure by an estimated 5%) and an increase in cerebral flow during diastole (occlusion of both femoral arteries will generate a second wave of flow during diastole and as a result diastolic pressure will increase by 10%). Since blood supply to the lower appendages will be reduced (by ˜20%) but will not be stopped, there will be no need for close medical supervision.

Aside from the therapeutic effects described above, the present method also provides numerous additional benefits to patients suffering from cardiovascular diseases.

Since cuff pressure is typically maintained above venous pressure, venous flow is stopped, and overall venous return during exercise per minute is reduced. This effect is of significant clinical importance to patients with chronic heart failure, since in such patients exercising increases venous return and left ventricular blood volume and as a result strain on the heart muscle increases. Since in such patients the heart muscle is not capable of increased force, their exercise capacity is significantly limited (Osada et al. 2003). Reduction of venous return will reduce heart muscle workload, and increase exercise capacity in patients suffering from cardiovascular disorders.

In addition, increased resistance due to cuff pressure on the non-exercised appendage will significantly reduce the blood flow to this limb and as a result, blood will flow to the exercised appendage which vasodilated due to exercise. This will increase the amount of oxygen that is delivered to the exercised appendage without further increasing cardiac output. Since in chronic heart failure/pulmonary patients, oxygen supply to the working muscle in a severe limitation, a better cardiac output distribution will improve exercise capacity (Osada et al. 2003).

Physiological responses to exercise in cardiovascular patients are different from those of healthy subjects. The main reasons for such differences are changes in artery function due to reduced arterial compliance, limited cardiac oxygen demand (which can result from drug therapy), and muscle de-conditioning that induces lower work capacity and moderate exercise responses.

Since the present method increases the exercise capacity of cardiovascular patients it indirectly contributes to benefits gained from such an increase in exercising.

Several studies (Ades et al. 1992, Evenson et al. 1998 and Witt et al. 2004) estimated the amount of physical activity needed to achieve a preventive effect. These studies found that in order to achieve desired improvements in physical fitness, aerobic exercise three times a week at a moderate intensity of 60 to 70% of the maximal heart rate is required. In order to improve coronary risk factor profile, patients should perform a moderate exercise at 55 to 65% of heart rate reserve, five times per week for 45 minutes.

Although exercise provides many benefits for cardiac patients, exercise programs suffer several limitations, including lack of therapeutic effects on the coronary arteries (20-21). This is due to the fact that most patients cannot reach the exercise intensity required for achieving a beneficial effect due to decreased cardiac output, orthopedic disorders or injury, neurological disorders, lack of motivation, anxiety, low physical fitness, angina pectoris, dispanea, peripheral vascular disease, valve problems, pulmonary disease, hypertension and arrhythmia.

Exercise training in patients with heart diseases at moderate intensity (3-5 times a week) leads to a marked improvement in peak exercise capacity after 8 to 10 weeks. Several reports have found an improvement of about 18 to 25% in peak oxygen consumption and an increase of 18 to 34% in peak exercise duration (Ades P A N Engl J. Med. 2001; 345:892-902 In patients with angina pectoris or silent ischemia, a much moderate improvement occurs. Subjective symptoms, activity profile and quality of life scores are also improved following exercise (Ades Cardiol Clin. 2003; 21:435-448).

During exercise, blood flow in the coronary arteries is increased and as result, shear stress on endothelial cells lining the blood vessels is increased. This leads to an intensified NO release to the blood circulation. NO molecules function in relaxation of smooth muscle cell of the arteries wall, vasodilatation of the arteries, and as anti adhesive molecules (Shephard et al. 1999). Several studies have found that exercise improves NO secretion and endothelial function, but those studies were performed on professional runners. Other studies performed on cardiac patients found that exercise can restore endothelial function of peripheral arteries. Only one study has evaluated the coronary endothelial function after a short (four weeks) intensive period of training (Hambrect R 2000). This study showed that intensive training improves endothelial function in patients with coronary artery disease.

It is also accepted that exercise acts as a stimulator for collateral formation (Laughlin, 2004).

Thus, the present method which improves coronary flow and increases the shear stress on blood vessel wall during exercise will improve the exercise therapeutic effect and make exercise more efficient, easier and safer for most cardiovascular patients.

The present method also enables subjects which can only exercise their upper body (subjects with physical limitations) to enjoy the therapeutic benefits typically only afforded by large muscle workouts (e.g. leg muscles).

As is mentioned hereinabove, the present method can be effected by a novel apparatus.

One embodiment of the apparatus of the present invention which is referred to herein as apparatus 10 is illustrate in FIG. 3a. A system which includes apparatus 10 as well as an exercise device is illustrated in FIG. 3b.

Apparatus 10 includes a control unit 12 which includes a mechanism 14 for deploying one or more cuffs 16 and preferably a user interface for controlling operation of apparatus 10. Mechanism 14 can be any mechanism capable of repeatedly deploying cuffs 16 through cycles of higher and lower pressure. Examples include: pressure 10-20 mmhg below the desired pressure, or 10 mmHg above the desired pressure.

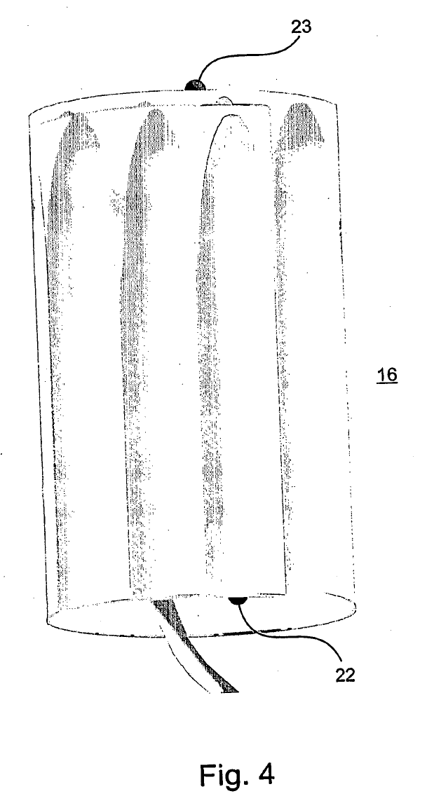

In a preferred configuration of apparatus 10, mechanism 14 includes an air pump and two inflatable/deflatable cuffs 16 (right and left). In such a configuration, cuffs 16 are connected through fluid communicating conduits 18 to mechanism 14 of control unit 12. Mechanism 14 may also include a valve 20 which can be manually operated or electromagnetically actuated by control unit 12. Cuffs 16 are designed such that during inflation, a pressure gradient forms along the width of the cuff from the distal end (e.g. in arm cuffs, end farther from shoulder) to the proximal end (e.g. 90 mm Hg at the distal end and 80 mm Hg at the proximal end). A pressure gradient-capable cuff 16 is illustrated in FIG. 4.

Cuffs 16 are preferably designed to be placed on the arms distal to the shoulder joint of the subject. Typical dimensions for such an arm mounted cuff 16 configuration are 15-20 cm in length and 30-50 cm in width. Cuff 16 can fabricated from any material including a polymer or a textile. Air pump configuration of mechanism 14 provides air under pressure through conduit 18, which can split into two separate conduits 18′ and 18″ each leading to a separate cuff 16. At least one of cuffs 16 includes a sensor 22 which is capable of directly or indirectly sensing blood pressure and relaying sensed information to control unit 12. Suitable sensor types include a temperature sensor, a plasmography sensor, or a microphone (e.g. piezo element). Sensor 22 is preferably a temperature sensor which can provide control unit 12 with temperature readings which can then be converted to pressure readings as is described in Example 1 of the Examples section which follows.

Cuffs 16 can be rapidly inflated and deflated any number of cycles and retained inflated for any time period. For example, cuffs 16 can be inflated to a pressure of e.g., 80 mmHg for at least five seconds. The function of cuff 16 in generating counterpulsation, especially in conjunction with exercising is described below.

Inflation of cuff 16 positioned on a non exercised limb to approximately 80 mm Hg during diastole will result in occlusion of arteries during diastole (pressure higher then diastolic) and thus ensure blood flow during systole. Use of sub-systolic pressure in cuff 16 ensures blood supply to the limb and no increase in cardiac oxygen demand.

During diastole, arterial pressure declines rapidly (e.g. 70 mm Hg), since pressure in cuff 16 is now higher then arterial pressure, the arteries in the limb will be occluded and the peripheral blood will flow towards the heart (this is facilitated by cuff 16 configuration shown in FIG. 4). As a result of the occlusion, the resistance to flow in the subclavian arteries/iliac arteries will increase and flow in those arteries will stop. Since aortic pressure during exercise is already elevated further increase in blood volume will cause a dramatic increase in aortic pressure (due to the fact that the aorta is a very elastic blood vessel) which will result in an increase of diastolic pressure in the aortic arch. Due to the fact that coronary resistance is reduced during diastole this pressure augmentation will increase coronary perfusion pressure and coronary flow.

Cuff 16 can be inflated/deflated at time interval ratios of 4 to 1 to 2 to 1, i.e. 20 second inflation and 5-10 seconds of deflation. The deflation period will enable venous flow, while during inflation pressure gradient of cuff 16. (of FIG. 4) will enhance venous flow. At full cuff inflation venous flow will stop (venous pressure is 30 mmHg).

When exercising, inflation of cuff(s) 16 above diastolic pressure causes several physiological modifications. During heart systole, as the pressure waves progress to the brachial/femoral arteries, the higher pressure inside the arteries e.g., 160 mm Hg exceeds the lower external pressure e.g., 90 mm Hg and ensures the brachial/femoral flow. The residue of the blood reaching the arms/legs will flow to the working muscles, where the resistance to flow is reduced. This will prevent the increase in systolic blood pressure, because the resistance to flow in the working extremities is decreased and the changes are minor compared to the increase in pressure in the aortic arch.

During inflation of Cuff 16, two factors influence the flow to the arms/legs, during diastole. Rapid decrease in the intra-artery pressure from 160 mm Hg to about 70 mm Hg, and occlusion of the brachial/femoral arteries due to external pressure.

These changes cause a small portion of back-flow towards the heart and as a result, a sharp increase in vascular resistance in subclavian/iliac arteries. The sharp alteration in vascular resistance will stop the flow to the subclavian/iliac arteries during diastole and will increase the pressure in the aortic arch. Due to the fact that only in the coronary circulation the resistance is reduced during heart diastole, more blood will flow to the coronary arteries and cause the effect of improving blood flow and increasing the coronary perfusion pressure. As a result, an increase in coronary artery wall shear stress occurs, and NO secretion is elevated thereby promoting angiogenesis.

Since during exercise diastolic pressure in the aortic arch is already increased, a further increase in pressure will cause a significant effect on coronary perfusion pressure due to the elastic properties of the arteries.

During inflation of Cuff 16, blood flow to the upper/lower extremities is reduced and causes ischemic metabolites accumulation. As a result a vasodilatory response is induced distal to Cuff 16. Cuff 16 deflation causes a short increase in flow to the arms/legs, as a result of reactive hyperemia. This will maintain the flow to those arteries and as a result, ensure the device's physiological effect.

Cuff deflation ensures venous return from the extremities to the heart, and during the inflation, cuff 16 compress the blood in the veins, as a result of the pressure difference between cuff 16 and the venous system.

Mechanism 14 preferably includes a pressure switch for stopping cuff deployment (e.g. inflation) when the pressure in cuffs 16 reaches a preset pressure calculated by the control unit 12 according to a measurement obtained by the sensor 14. Mechanism 14 renews operation when the pressure in cuffs 16 drops below the pressure that has to be maintained or when the deflation period is over and cuffs 16 have to be re-inflated.

The pressure regulator of the control unit 12 coordinates the pressure applied by the cuffs 16 with the pressure measured in the brachial/femoral artery. The pressure regulator sets the amount of air that flows into cuffs 16 according to the diastolic pressure/diastolic flow measurements. The pressure that has to be applied on the brachial/femoral artery is the diastolic pressure plus about 10 to 30 mm Hg and also depends on the program or manual control that suits the patient's clinical and physical conditions. The pressure regulator is operated automatically, but can be set manually by data input of the desired pressure that to be applied on the brachial/femoral artery, as set by a pressure selector of control unit 12.

The pressure regulator can also enable inflation/deflation of cuffs 16 at a rate that enables the operator to measure the blood pressure manually, e.g., by a stethoscope or automatically by sensor 22.

Control unit 12 is capable of receiving the data from sensor 22 and calculating the pressure applied in cuffs 16. Preferably, control unit 12 executes a software application which employs a specific mathematical integral. The integral is based on the changes of skin temperature above the brachial or femoral arteries/pulse sound/flow during cuff inflation/deflation. The software application can also be configured capable of calculating heart rate using ECG electrodes 23 implanted in cuff 16 and displaying data on a monitor connected to apparatus 10.

The software application of control unit 12 is designed capable of stopping inflation of cuffs 16 in several cases: sharp increase or decrease in diastolic pressure/flow, constant increase in the diastolic or systolic pressure in 5 to 6 consecutive measurements, without increase in heart rate, and in cases such as arrhythmias or arrest in ECG.

The software application of control unit 12 is designed for continuously integrating between the pressure and pulse samples and is capable of reducing the pressure applied in the cuffs (by e.g., 10-30 mmHg, in response to several exercising situations such as: increase/decrease in heart rate (Sinus rhythm) by 10 beats per minute, increase in heart rate above 140 or 2.5 folds from the exerciser's heart rate during rest, or decrease in the exerciser's heart rate to resting values.

The user interface of control unit 12 can include a program selector which employs a timer for controlling the duration of the applied pressure in cuffs 16, a pressure regulator 26 for setting cuff 16 pressure ranges or limits and a display 28 for providing the user with information such as blood pressure, cuff inflation pressure, heart rate, duration of treatment and the like. Inflation and deflation cycle times can be set by an operator and can be changed throughout exercising.

Sensor 22 is preferably located on or in cuffs 16, and is selected capable of measuring the changes in skin temperature/diastolic pressure/flow in an artery. and transferring the data to control unit 22 for calculating the pressure applied to the cuffs during inflation and deflation.

Apparatus 10 can also form a part of an exercise system, which is referred to herein as system 100.

System 100 includes apparatus 10 which is attached to, or integrated with an exercise apparatus 102. Exercise apparatus 102 can be any physical exercise apparatus, several examples of which are provided hereinabove.

When utilized in combination with, or as apart of an exercise system, apparatus 10 also includes software and hardware required for controlling the operation of Exercise apparatus 102. For example, control unit 12 includes hardware for interfacing with sensors mounted in or on exercise apparatus 102, which sensors provide data on level of exercising etc.

It is expected that during the life of this patent many relevant pressure devices will be developed and the scope of the phrase “device designed capable of periodically reducing blood flow in at least one appendage” is intended to include all such new technologies a priori.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Cuff Pressure as Measured by a Cuff Temperature Sensor

Experiment Model

A temperature sensor (9707280 Betabeam USA) was connected to laboratory made amplifier custom made see the attached figure. The amplifier was connected to personal computer and the recoded sensor signal was processed using MatLab software (Version 2004). A pressure sensor that indicates cuff pressure was also connected to the computer and was used to correlate temperature readings with pressure.

Experiment Protocol

The temperature sensor was attached to a skin region of an upper arm of the subject and a blood pressure cuff of an automatic blood pressure device (Omeron, Japan) was placed over the sensor. The blood pressure device was operated and changes in temperature and pressure were recorded simultaneously; measurements were repeated 15 times.

Results

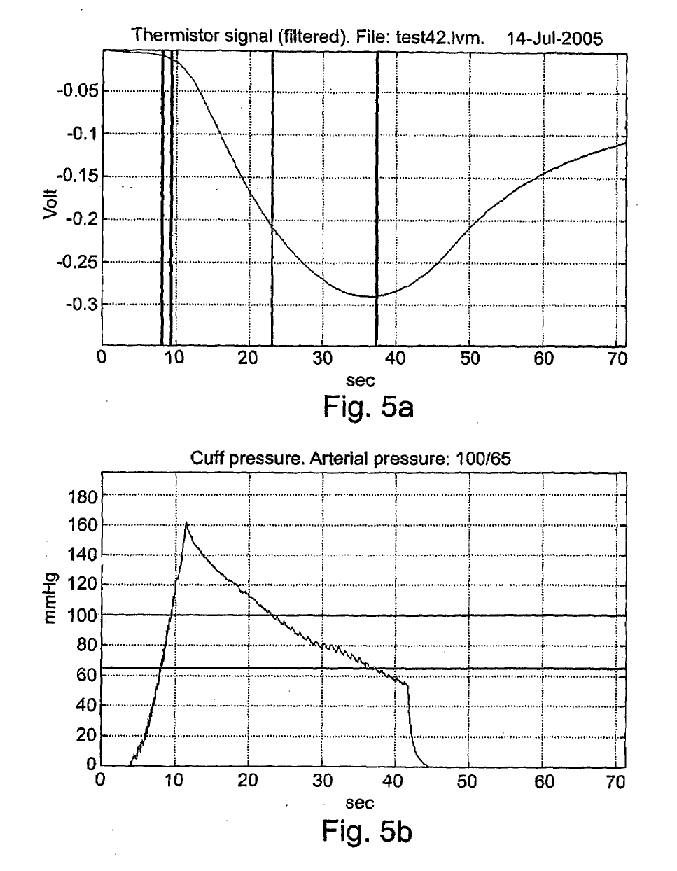

Typical plot of the correlation between pressure applied by the cuff and temperature changes is shown in FIGS. 5a-b. As the cuff reached a pressure above systolic pressure, skin temperature responses declined sharply. During cuff deflation a moderate temperature decline was observed for 3-4 seconds. This decline in temperature continued until cuff pressure reached the diastolic pressure, at that point the temperature started to rise due to an increase in blood flow.

Thus, the present study shows that a temperature-response graph of an inflation-deflation cycle of a pressure cuff can be used to monitor and determine systolic and diastolic pressures proving that a skin temperature sensor can be used to monitor systolic and diastolic pressure and control cuff pressure in an ECP procedure.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

1. Bouki K P, Pavlakis G, Papasteriadis E. Management of cardiogenic shock due to acute coronary syndromes. Angiology. 2005 March-April; 56(2):123-30.
2. Bonetti P O, Holmes D R Jr, Lerman A, Barsness G W Enhanced external counterpulsation for ischemic heart disease: what's behind the curtain? J Am Coll Cardiol. 2003 Jun. 4; 41(11):1918-25
3. Gaenzer H, Neurnayr G, Marschang P, Sturm W, Kirchmair R, Patsch J R. Flow-mediated vasodilation of femural and brachial artery induced by exercise in healthy nonsmoking and smoking men. J Am Coll Cardiol. 2001; 38(5):1313-9IL.
4. Piña, C S. Apstein, G J. Balady, et al. Exercise and Heart Failure A Statement From the American Heart Association Committee on Exercise, Rehabilitation, and Prevention Circulation. 2003; 107:1210
5. Osada T, Katsumura T, Murase N. Post-exercise hyperemia after ischemic and non-ischemic isometric handgrip exercise. J Physiol Anthropol Appl Human Sci. 2003; 22(6):299-309.

6. T Hamada, T Hayashi, T Kimura, K Nakao, T Moritani. Electrical stimulation of human lower extremities enhances energy consumption, carbohydrate oxidation, and whole body glucose uptake. J Appl Physiol 96: 911-916, 2004
7. Panerai R B, Dawson S L, Eames P J, Potter J F. Cerebral blood flow velocity response to induced and spontaneous sudden changes in arterial blood pressure. Am J Physiol Heart Circ Physiol. 2001; 280(5):H2162-74
8. Kashyap V S, Franklin K S, Fitzpatrick C M. Bilateral brachial artery occlusion decreases internal carotid artery volume flow: a simple adjunct for cerebral protection?. J Endovasc Ther. 2005 August; 12(4):454-60.
9. Cardiac rehabilitation and secondary prevention of coronary heart disease. N Engl J Med. 2001; 345:892-902
10. Leon A S, et al. Cardiac rehabilitation and secondary prevention of coronary heart disease. Circulation. 2005; 111:369-376.
11. Ades P A, et al. Effects of exercise and cardiac rehabilitation on cardiovascular outcomes. Cardiol Clin. 2003; 21:435-448.
12. Laughlin M H. Physical activity in prevention and treatment of coronary disease: the battle line is in exercise cell biology. Med Sci Sports Exer. 2004; 36:352-362.
13. Lerman A, et al. Endothelial function: cardiac events. Circulation. 2005; 111:363-368.
14. Shephard R J, et al. Exercise as cardiovascular therapy. Circulation. 1999; 99:963-972.
15. Hofman-Bang C, et al. Two-year results of a controlled study of residential rehabilitation for patients with percutaneous transluminal coronary angioplasty. Am Heart J. 1999; 201465-1474.
16. Hosokawa S, et al. Effect of regular exercise on coronary endothelial function in patients with recent myocardial infarction. Circulation. 2003; 67:221-224.
17. Ades P A, Waldmann M L, McCann W J, Weaver S O. Predictors of cardiac rehabilitation participation in older coronary patients. Arch Intern Med. 1992 May; 152(5):1033-5.
18. Evenson K R, Rosamond W D, Luepker R V. Predictors of outpatient cardiac rehabilitation utilization: the Minnesota Heart Surgery Registry. J Cardiopulm Rehabil. 1998 May-June; 18(3):192-8.
19. Witt B J, et al. Cardiac rehabilitation after myocardial infarction in the community. J Am Coll Cardiol. 2004; 44:988-996.
20. Centers for Disease Control and Prevention (CDC). Receipt of cardiac rehabilitation services among heart attack survivors—19 states and the District of Columbia, 2001. MMWR Morb Mortal Wkly Rep. 2003 Nov. 7; 52(44):1072-5.
21. Fromy B, Abraham P, Bouvet C, Bouhanick B, Fressinaud P, Saumet J L Early decrease of skin blood flow in response to locally applied pressure in diabetic subjects. Diabetes 51:1214-1217, 2002
22. van Marum R J, Meijer J H, Bertelsmann F W, Ribbe M W. Impaired blood flow response following pressure load in diabetic patients with cardiac autonomic neuropathy. Arch Phys Med Rehabil. 1997 September; 78(9):1003-6.

What is claimed is:

1. An apparatus for improving heart perfusion of a subject comprising:
(a) at least one device designed capable of periodically reducing blood flow in at least one appendage of the subject; and
(b) a control unit being capable of calculating flow in peripheral arteries and being for controlling an operation of said at least one device according to said flow in said peripheral arteries.

2. The apparatus of claim 1, wherein said at least one device is an inflatable cuff.

3. The apparatus of claim 1, wherein said at least one device is designed for use on an arm of the subject.

4. The apparatus of claim 1, wherein said at least one device includes an electrode for electrically stimulating contraction of a muscle of said at least one appendage.

5. The apparatus of claim 1, wherein said at least one device includes a sensor for measuring blood flow and whereas said control unit is capable of calculating flow changes in peripheral arteries according to data received from said sensor.

6. An apparatus for improving heart perfusion of a subject comprising:
(a) at least one device designed capable of periodically reducing blood flow in at least one appendage of the subject;
(b) a temperature sensor being for measuring a temperature of said at least one appendage; and
(b) a control unit for controlling an operation of said at least one device according to temperature data received from said temperature sensor.

7. The apparatus of claim 6, wherein said temperature sensor is integrated with said at least one device designed capable of periodically reducing blood flow to said at least one appendage of the subject.

8. The apparatus of claim 6, wherein said at least one device is an inflatable cuff.

9. The apparatus of claim 6, wherein said at least one device is designed for use on an arm of the subject.

10. The apparatus of claim 6, wherein said at least one device includes an electrode for electrically stimulating contraction of a muscle of said at least one appendage.

11. A method of improving heart perfusion comprising:
(a) periodically reducing blood flow in at least one appendage of a subject; and
(b) subjecting said subject to physical exercise, thereby improving heart perfusion of said subject.

12. The method of claim 11, wherein said at least one appendage is an arm.

13. The method of claim 12, wherein said physical exercise is walking or running.

14. The method of claim 11, wherein (a) is effected by applying to said at least one appendage a pressure higher than a diastolic pressure of said subject for a predetermined period of time.

15. The method of claim 11, wherein (a) and (b) are effected simultaneously.

16. A system for improving heart perfusion of a subject comprising:
(a) at least one device designed capable of periodically reducing blood flow in at least one appendage of the subject;
(b) an exercise unit for subjecting said subject to physical exercise;
(c) a control unit for controlling an operation of said at least one device and said exercise unit.

17. The system of claim 16, said at least one device is an inflatable cuff.

18. The system of claim 16, wherein said at least one device is designed for use on an arm of the subject.

19. The system of claim 16, wherein said at least one device includes an electrode for electrically stimulating contraction of a muscle of said at least one appendage.

20. The system of claim 16, wherein said exercise unit is selected from the group consisting of a stationary bicycle, a treadmill and an arm ergometer.
